(19) United States
(12) Patent Application Publication
(10) Pub. No.: US 2012/0029499 A1
(43) Pub. Date: Feb. 2, 2012

(54) METHOD AND APPARATUS FOR PLASMA SKIN RESURFACING

(75) Inventor: Gian Franco Bernabei, Florence (IT)

(73) Assignee: MATTIOLLI ENGINEERING LTD.

(21) Appl. No.: 13/132,853

(22) PCT Filed: Jul. 6, 2009
(86) PCT No.: PCT/IB2009/006166

§ 371 Date: Oct. 13, 2011

Related U.S. Application Data
(60) Provisional application No. 61/078,667, filed on Jul. 7, 2008.

(51) Int. Cl. A61B 18/14

(52) U.S. Cl. 606/33

(57) ABSTRACT

An apparatus for treating a skin surface of a patient includes a probe having an opening to be in contact with the skin surface. The probe includes an electrode disposed within the probe and connected to a coaxial cable, the electrode configured to receive radio frequency power and to provide a glow discharge when a vacuum is provided to the probe. A shield is provided between the skin surface and the probe, whereby the shield includes a plurality of holes, and whereby ions of a plasma discharge pass through the holes of the shield and impact the skin surface.

### BACKGROUND OF THE INVENTION

1. Field of the Invention

The present invention relates to a method and apparatus for applying to the skin, in a controlled manner, a radio frequency generated plasma in order to heat and selectively damage thin superficial layers of the skin, thereby inducing a renewal process of the epidermis.

2. Description of the Related Art

It is well known in the skin treatment art that in order to renew the epidermis layer, induced damage of the skin is required. One such method uses laser radiation that is incident on the skin and that generates several effects on the skin, depending on the wavelength of the laser radiation, the pulse duration of the laser energy applied to the skin, and the radiation energy provided to the skin.

The most commonly used method is CO₂ laser radiation for generating a superficial heating of the skin. When laser light reaches the skin, its intensity decreases exponentially as it progresses down into lower layers of the skin. This means that the thermal energy that is delivered is higher in the first layer and decreases exponentially as its progresses down to lower layers of the skin. Moreover, the first corneum stratus of the skin has a higher absorption than other layers. Such an energy profile is not suitable for a uniform heating of a volume of skin due to the fact that in the superficial (upper) layers, the reached temperature is too high and in the lower layers the reached temperature is not high enough to trigger the desired skin treatment process.

Two principles are used in U.S. Pat. No. 6,518,538, which is incorporated in its entirety herein by reference. First, radio frequency currents are localized in the external layer of the skin due to the skin effect, and thus the heating is localized in a thin (upper) layer of skin.

It is well known that an alternating voltage applied to a conductor generates a current on the external layer of the conductor and the depth depends on the frequency and the resistance of the conductor (so-called skin effect).

Second, the plasma generated at the contact of the skin, due to the radio frequency and a high vacuum generated by a suitable pump, is composed of high energy gas ions that strike the surface of the skin, thereby generating heat in the superficial layer of the skin.

The interaction with the skin has some similarities to the interaction described in U.S. Pat. No. 6,269,271, which is incorporated in its entirety herein by reference.

One advantage of such an approach is by not having electrodes in contact with the skin, a more even distribution of the radio frequency current in the skin is achieved. Also, there is achieved a combined action from the striking gas ions and a more accurate control of the power applied to the skin surface, due to the higher impedance of the plasma that controls the current independently from the electrical conductivity value of the skin.

U.S. Pat. No. 6,518,538 describes an apparatus and a method for skin resurfacing treatment, which provides induced thermal damage of the skin by radio frequency heating and by ion bombardment of the skin.

In U.S. Pat. No. 6,518,538, this dual effect may be achieved by using a pulsed radio frequency generator connected to a probe for coupling to the skin. The probe is preferably made of a non-conductive material (such as glass or plastic), and enables the application of a high vacuum to the skin surface (e.g., 5-10 millibars) over a predetermined (e.g., round) portion of the skin, by using a non-conductive pipe connected to a vacuum pump. At a suitable distance (around 10 millimeters) from the surface of the skin, an electrode (that is housed within the probe) is used to generate a radio frequency field between the electrode itself and the surface of the skin. After reaching a sufficient vacuum (e.g., 5-10 millibars of atmospheric pressure), a high voltage radio frequency electric field is applied between the electrode and the surface of the skin, due to a radio frequency pulse applied to the electrode. Such a radio frequency field triggers a glow discharge inside the probe between the electrode and the skin. A radio frequency current, due to the low impedance of the glow discharge, flows evenly on the surface of the skin, and, due to the skin effect, is limited to the glow discharge area in a depth of about 300 microns. In the surrounding tissues, the current density decreases by the square of the distance from the area covered by the glow discharge within a depth of 300 microns. Moreover, the high energy ions of the glow discharge strike the surface of the skin, thereby providing a plasma skin resurfacing that can be used to remove spider veins, skin brown spots, or port wine stains, for example.

U.S. Pat. No. 6,518,538 describes the providing of a controlled heating of a selected portion of the skin to a depth of about 300 microns. As a result, it is possible to reach a desired temperature of 70 degrees C. or more, which triggers controlled damage to the skin cells to achieve a desired effect. The temperature reached in the described volume of the skin depends primarily on the selected pulse length and the power of the radio frequency generator. Preferably, a temperature reached in the described volume of the skin is a temperature in the range of from 75 degrees C. to 95 degrees C.

To achieve a substantially uniform heating of a volume of the skin, a method according to U.S. Pat. No. 6,518,538 includes:
1) Application of a probe to the skin, where the probe is held against an open area on the skin of about one square centimeter, where the probe includes an electrode at a distance of 10 millimeters (plus or minus a few millimeters) from the skin surface, and where a vacuum suction pipe is connected to the probe.
2) Generation of a high vacuum inside the probe and at the surface of the skin by connection of the probe to a high vacuum pump, by way of the vacuum suction pipe.
3) Application of high voltage at a frequency of 21 MHz in the probe between the electrode and the skin, by way of a pulsed radio frequency generator connected to the probe by way of a conductive cable.
4) Generation of a glow discharge for a time less than 1 second sustained by a power less than 500 W.

One problem associated with the apparatus and method described in U.S. Pat. No. 6,518,538 is that the skin is sucked into the probe when the diameter of the probe is larger than 10 millimeters, which is an undesirable occurrence.

Another problem associated with the apparatus and method described in U.S. Pat. No. 6,518,538 is that the large current in the plasma could create some damage to the skin, while the skin is being treated during a skin resurfacing procedure.

### SUMMARY OF THE INVENTION

The present invention utilizes a method and apparatus of heating a superficial portion of skin using a combined action of radio frequency and a plasma generated by the same radio frequency, while also using a shield that is provided between the probe (that performs the plasma skin resurfacing procedure) and the skin.

### BRIEF DESCRIPTION OF THE DRAWINGS

The invention will become more fully apparent from the following detailed description when read in conjunction with the accompanying drawings with like reference numerals indicating corresponding parts throughout, and wherein:

FIG. 1 shows a system that may be utilized to treat a skin surface in order to provide relatively uniform skin heating, in accordance with a first implementation of a first embodiment of the invention;

FIG. 2 shows a system that may be utilized to treat a skin surface to provide relatively uniform skin heating, in accordance with the first embodiment of the invention;

FIG. 3 shows a system that may be utilized to treat a skin surface, in accordance with a second implementation of the first embodiment of the invention;

FIG. 4 shows a front view of one possible implementation of a shield that is used in a system in accordance with the first embodiment of the invention;

FIG. 5 shows a system that may be utilized to treat a skin surface in order to provide relatively uniform skin heating, in accordance with a second embodiment of the invention; and

FIG. 6 shows a front view of one possible implementation of a shield that is used in a system in accordance with the second embodiment of the invention.

### DETAILED DESCRIPTION OF THE PREFERRED EMBODIMENTS

Preferred embodiments of the present invention will be described in detail hereinbelow, with reference to the drawings.

According to the present invention, a probe is put in contact with the skin to be treated (e.g., so as to remove spider veins or brown spots or port wine stains from the skin surface, for example), whereby a shield is also provided between the probe and the skin. The shield operates to limit the amount of current in the plasma applied to the skin, so as to limit the amount of any damage that may occur to the skin. The shield also operates to block the skin from being sucked into the probe, which would otherwise cause an unwelcome and unnecessary pain to the patient being treated.

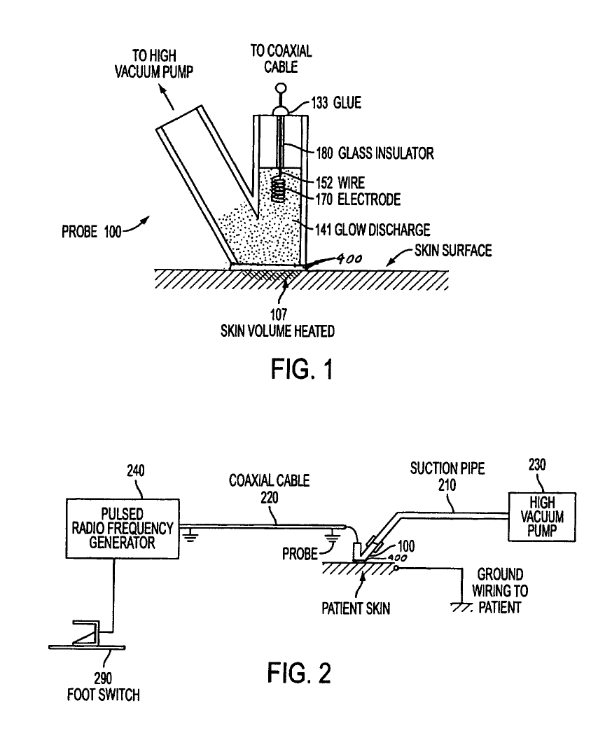

In a first implementation of a first embodiment of the invention, as seen in FIG. 1, the probe 100 is V-shaped and is preferably made from polycarbonate. However, other types of plastic materials or glass or suitable insulating material may be used for the probe 100. Referring now to FIGS. 1 and 2, a first upper end of the V-shaped probe 100 is connected to a vacuum suction pipe 210, and a second upper end of the V-shaped probe 100 is connected to a coaxial cable 220. The coupling of the vacuum suction pipe 210 to the first upper end of the probe 100 and the coupling of the coaxial cable 220 to the second upper end of the probe 100 are air-tight couplings. That way, a vacuum can be formed within the probe 100. The bottom end of the V-shaped probe 100 has an opening that is to be placed in direct contact with a portion of the skin to be treated (shown as cross-hatched area 107 in FIG. 1), to provide an air-tight coupling of the opening against the skin surface. A shield 400 is attached to the probe 100, whereby the shield 400 is described in detail in a later portion of this specification.

The opening of the probe 100 preferably has a smooth round edge in order to assure a tight coupling with the skin and to avoid vacuum leakage. The opening is preferably round in shape, but any other shape can be used. In the first embodiment, the opening has a diameter of 8 millimeters, but other sizes may be utilized while remaining within the scope of the invention. For example, a larger diameter opening may be used by increasing the stroke of the vacuum pump 230, the diameter of the suction pipe and the power of the radio frequency generator 240. The power of the radio frequency generator 240 should be increased linearly with the increase of the surface covered by the glow discharge, in order to obtain substantially the same temperature on the skin.

The first upper end of the V-shaped probe 100 is connected to the coaxial cable 220 by way of a glass insulator 180 fed through to the probe 100. The glass insulator 180 covers one end of the coaxial cable 220 that is coupled to the probe 100. A copper wire 152 is incased within the glass insulator 180, and is preferably welded to a terminal end of an inner wire of the coaxial cable 220.

In case of feeding of gas, as in a second implantation of the first embodiment (see probe 100′ in FIG. 2) to be described later, the upper part of the probe is modified in order to enable a flow of gas between the copper wire and the glass insulator. Glass is used instead of plastic for the wire insulator within the probe, due to the high temperature that the electrode reaches during the operation of the probe for treating a patient's skin. Other materials, such as ceramic, could be used as well. A suitable glue 133 is used in order to assure that the vacuum is tight and that no leaks occur between the copper wire 152 and the glass insulator 180 (at the top portion of the probe 100 in the view of FIG. 1).

In the first implementation of the first embodiment, an electrode 170 is formed at a distal end of the copper wire 152, where the copper wire is wound by several turns with a diameter of about 1 millimeter for each of the turns, thereby forming the electrode 170. For example, five turns are used in the first embodiment, but other numbers of turns, as well as turn diameters, may be used while keeping within the scope of the invention. A glow discharge emanates from the electrode 170 when subject to pulsed radio frequency energy. The electrode 170 is disposed within the probe 100 in such as manner as to not be in contact with either the walls of the probe 100 or the surface of the skin. As explained above, the copper wire 152 is fitted inside the glass insulator 180 and is connected with an inner conductor (wire) of the coaxial cable 220, so as to receive radio frequency energy from the radio frequency pulse generator 240 by way of the coaxial cable 220.

The distance between the last turn of the electrode 170 (that is furthest from the coaxial cable 220) and the bottom opening of the V-shaped probe 100 is preferably 10 millimeters. That range may be varied (e.g., 5-20 mm range, for example) to provide a desired temperature to the skin. The positioning of the turns of the electrode 170 and the copper wire 152 is such that the turns are orthogonal to the surface of the opening, in order to have an even distribution of the electric field as it impinges on the surface of the skin.

The first upper end of the V-shaped probe 100 is connected through the suction (or vacuum) pipe 210 to the high vacuum pump 230. In the first embodiment, an oil rotary pump is used which can provide up to a 5 millibar vacuum.

In the first implementation of the first embodiment, the coaxial cable 220 has a length of 2.3 meters, and is used as an impedance transformer from the low impedance output of the radio frequency generator 240 (52 ohm) to the probe 100, to provide for a glow discharge at a desired (e.g., 21 MHz) frequency. Other cable length are suitable at different frequencies and with other types of radio frequency generators, as well as high voltage radio frequency transformers.

The radio frequency generator 240 used in the present invention may be a conventional power generator having a pulse duration that is selectable, and having an output power capability of up to 500 W. The triggering of a pulse may be done by a footswitch 290, for example, or by other ways (e.g., toggle switch on the housing of the radio frequency generator 240). A preferred pulse duration is a value of between 1 milliseconds and 1000 milliseconds. An output power of the radio frequency generator 240 may be between 1 and 500 W, depending on the desired temperature to which the skin surface is to be heated. Also, the output radio frequency may be a value within the range of between 2 MHz and 52 MHz. Upon selecting a different frequency, the depth of the heated volume of the skin by the radio frequency current vary, i.e., the higher the frequency, the less the depth. The cable length of the coaxial cable 220 is chosen in order to match the high impedance of the glow discharge with the low impedance of the radio frequency pulse generator 240, and is approximately one-fourth of the wavelength of the radio frequency traveling inside the coaxial cable 220.

When the probe 100 is placed in contact with a desired area of a patient's skin to be treated, the vacuum pump 230 is activated. Upon reaching a vacuum pressure of 10 millibars or less, the footswitch 290 is then activated, thereby enabling the generation of the radio frequency voltage. The radio frequency voltage travels along the coaxial cable 220 to the electrode 170, whereby a glow discharge is generated due to the vacuum within the probe 100. The glow discharge within the probe 100 is shown as the gas-like region 141 in FIG. 1. As seen in FIG. 2, the patient is preferably grounded, to enhance the attraction of the gas ions within the glow discharge to the patient's skin.

Radio frequency current as well gas ions are applied to the surface of the skin under the opening of the probe 100. Gas ions of the glow discharge act as a conductor, enabling the flow of current. When the gas ions strike the surface of the skin at high speed, they penetrate inside and they lose their charge, thus enabling the flow of current.

The frequency generator 240 is switched off after the pre-selected pulse width of radio frequency energy has been applied to the probe 100. This enables the reaching of a desired superficial temperature of the skin, so as to generate a desired amount of heat damage of the skin cells under the probe 100 (so as to remove port wine stains or spider veins or skin brown spots, for example).

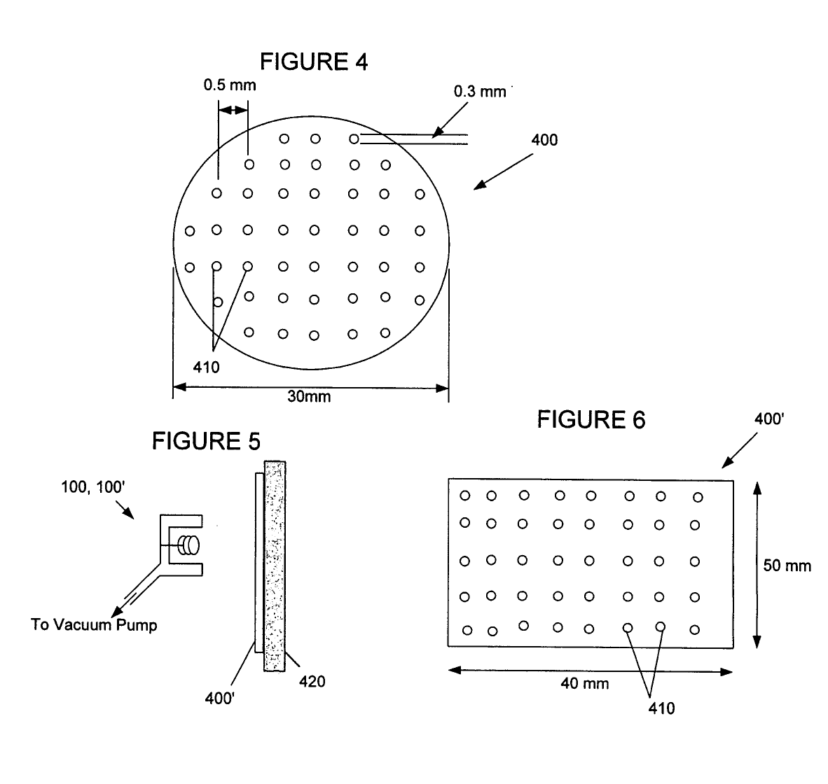

Together with the probe described in detail above, the first embodiment utilizes a shield that is provided between the probe and the skin, whereby the shield is directly attached to the probe. The shield has small holes provided throughout its surface. The shield may be conductive (e.g., stainless steel) or non-conductive (e.g., polyester or plastic). In one possible implementation of a shield 400 that can be used in a skin treatment system according to the first embodiment, as seen in FIG. 4, each of the holes 410 provided in the shield 400 has a diameter of 0.3 mm, whereby a distance between the centers of adjacent holes is 0.5 mm. Of course, other sizes and hole spacings may be contemplated while remaining within the spirit and scope of the invention. The holes 410 of the shield 400 are positioned directly underneath the opening of the probe 100 that that outputs the plasma to a portion of the skin to be treated. The density of the holes 410 provided in the shield is 400 holes/cm² in one possible implementation of the present invention, whereby other densities (e.g., 200 to 600) may be contemplated while remaining within the spirit and scope of the invention (FIG. 4 shows a much lesser density of holes for purposes of clarity in order to clearly show the spacings between holes and the sizes of the holes). In a preferred configuration, the shied 400 is sized to fit right over the plasma output hole of the probe 100, and thus is sized to be of a comparable diameter as the plasma output hole of the probe 100. The shield 400 is thin, with a 1 mm thickness in one possible implementation of the first embodiment. As seen in FIG. 4, the shield 400 is circular in shape with a 30 mm diameter, so as to be sized to accommodate a 30 mm in diameter plasma output opening of the probe 100.

If the shield is made out of conductive material, such as stainless steel, the plasma discharge during skin treatment with the probe occurs between the central electrodes of the probe 100 and the shield 400, with the holes 410 of the shield 400 being electrically connected to ground. The ions of the plasma discharge go through the holes 410 of the shield 400, and impact to the skin, thereby generating heat. The result is a controlled damage of the epidermis of the skin, whereby this controlled damage triggers the formation of new collagen for the skin.

If the shield 400 is made out of non-conductive material, such as polyester or plastic, the plasma discharge occurs as before between the central electrodes and the shield 400 as described above with respect to a shield made out of conductive material, but due to capacitive coupling, a radiofrequency current is also generated in the skin, whereby the ions of the plasma discharge go through the holes 410 of the shield 400 and impact the skin, thereby generating further heat.

Furthermore, the shield (whether made or conductive material or non-conductive material) 400 enables the treating of a large area of skin (e.g., 30 mm×30 mm) without causing any problem with respect to suction of the skin, because the shield 400 operates to keep the skin directly under the probe flat, and thereby the skin is not sucked into the opening of the probe 100. The holes 410 of the shield 400 are sufficiently small so that the skin is not sucked into those holes. By way of example, for a probe having a 10 mm diameter opening for outputting plasma to a skin surface, the shield provided for the prove has 300 small holes and provides for a power limit of 500 watts, and by way of example, for a probe having a 30 mm diameter opening, the shield has 2700 small holes and provides for a power limit of 4500 watts.

Accordingly, better control of current to the skin during a skin treatment is obtained by using a shield along with the probe, as described above, and also the skin is kept from being sucked into openings of the probe.

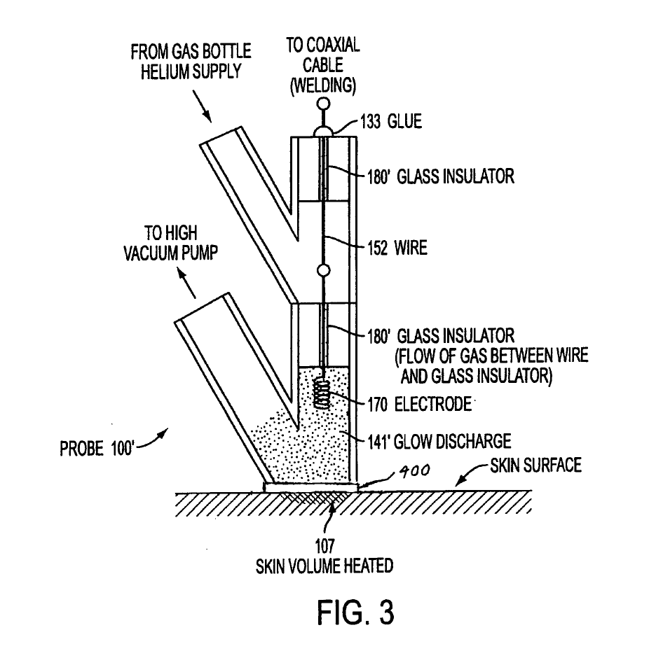

In a second implementation of the first embodiment, as shown in FIG. 3, a supply of low pressure gas, such as Helium, is provided to a third input port of a probe 100′ (which has similar features in other respects to the probe 100 shown in FIG. 1) in order to maintain a gas of controlled composition at a desired vacuum pressure (e.g., 10-50 millibars) over the skin. This low pressure gas is provided by a gas source (e.g., external canister of gas) that feeds the gas through an additional (third) input port of the probe 100′. As in the first implementation of the first embodiment, the first input port of the V-shaped probe 100′ is connected to the radio frequency pulse generator 240 by way of a coaxial cable 220, and the second input port of the V-shaped probe 100′ is connected to the vacuum source 230 by way of the vacuum pipe 210, to thereby provide a vacuum or near-vacuum condition within the probe 100′. In the second implementation of the first embodiment, the glass insulator 180′ has an opening to expose a portion of the copper wire 152 to the flow of helium gas supplied from the third input port of the probe 100′. This enables a flow of gas between the copper wire 152 and the glass insulator 180′, to provide a more stable glow discharge within the probe 100′. Similar to the first implementation of the first embodiment, a shield 400 is directly attached to the plasma output opening of the probe 100′.

In this second implementation of the first embodiment, the low pressure gas is supplied at a pressure of between 10-50 millibars, in order to stabilize the glow discharge and to selectively inject ions in the skin. Other gases besides Helium may be utilized while remaining within the scope of the invention, for example, Nitrogen or Oxygen or mixtures of gas including Helium may be used instead of Helium only.

Like the first implementation of the first embodiment, a shield 400 is utilized between the probe 100′ and the skin in the second implementation of the first embodiment, in order to limit the amount of current applied to the skin and to keep the skin from being sucked into the plasma output opening of the probe 100′. Thus, in the first embodiment, as the probe 100, 100′ is moved across the skin surface, the shield 400 moves with the probe 100, 100′.

The attachment of the shield 400 to the probe 100, 100′ may be a permanent attachment, or alternatively it may be a releasable attachment, to thereby allow for removal of the shield 400 from the probe 100, 100′ for cleaning of the shield 400, or for attaching a new shield for a later skin treatment using the same probe 100, 100′. To accomplish the releasable attachment, attachment means is provided on the probe 100, 100′, whereby the attachment means may include snap fit connection components, VELCRO™, rubber band, screws, or other types of connections that allow for the shield to be easily attached to and detached from the probe 100, 100′. To accomplish the permanent attachment, the shield 400 may be glued or otherwise affixed to the probe 100, 100′.

FIG. 5 shows a side view of a skin treatment system according to a second embodiment of the invention, in which a shield 400′ is sized to be much larger than the plasma output opening of the probe 100, 100′. In the second embodiment, the shield 400′ is not directly attached to the probe 100, 100′, but rather the shield 400′ is placed against the patient's skin 420, and the probe 100, 100′ is moved across the shield 400′ so as to treat a specific area of the patient's skin covered by the shield 400′. The shield 400′ is shown to have a size of 40 mm×50 mm, whereby the density of the holes provided on the shield 400′ is similar to that described above with the first embodiment (e.g., 400 holes/cm²). Referring now to FIG. 6, which shows a front view of the shield 400′, the holes 410 of the shield 400′ are provided throughout the entire surface of the shield 400′.

While the present invention has been described with respect to the preferred embodiments, other types of configurations may be possible, while remaining within the spirit and scope of the present invention, as exemplified by the claims.

What is claimed is:

1. An apparatus for treating a skin surface of a patient, comprising:
a probe having an opening to be in contact with the skin surface;
an electrode disposed within the probe and connected to a coaxial cable, the electrode configured to receive radio frequency power and to provide a glow discharge when a vacuum is provided to the probe; and
a shield provided between the skin surface and the probe, the shield including a plurality of holes,
wherein ions of a plasma discharge pass through the holes of the shield and impact the skin surface.

2. The apparatus according to claim 1, wherein the glow discharge provides a substantially uniform heating of the skin surface down to at least a predetermined depth beneath the skin surface.

3. The apparatus according to claim 1, further comprising:
a radio frequency generator that provides a radio frequency voltage;
a vacuum pump that provides the vacuum;
a suction pipe connected between the vacuum pump and the probe, the suction pipe providing the vacuum to the probe via a first input port of the probe; and
a coaxial cable that provides the radio frequency voltage to the probe via a second input port of the probe.

4. A method for treating a skin surface, comprising:
controlling a pulsed radio frequency generator to output at least one pulse;
controlling a vacuum source to provide a vacuum;
providing the at least one pulse and the vacuum to a probe to be provided directly on the skin surface to be treated, the probe having an opening that covers the skin surface to be treated, the probe further having an electrode which receives the least one pulse and which is under vacuum due to the vacuum provided by the vacuum source; and
providing a shield between the probe and the skin surface to be treated,
wherein ions of a plasma discharge pass through the holes of the shield and impact the skin surface.

5. The method according to claim 4, wherein a glow discharge is provided to the skin surface as a result, in order to provide a substantially uniform heating of the skin surface and regions below the skin surface to a fixed depth therebelow.

6. The method according to claim 4, wherein the at least one pulse has an output power of between 1 and 500 W, an output frequency of between 2 MHz and 52 MHz, and an output pulsewidth of between 1 and 1000 millisecond

7. The method for treatment of a skin surface according to claim 4, wherein treatment is to remove unwanted brown spots from the skin surface.

8. The method for treatment of a skin surface according to claim 4, wherein low pressure Helium is injected in the glow discharge.
